(19) United States
(12) Patent Application Publication
(10) Pub. No.: US 2013/0018350 A1
(43) Pub. Date: Jan. 17, 2013

(54) Disposable Incontinence Pouch Assembly

(71) Applicant: Edward McNulty, Midland, VA (US)

(72) Inventor: Edward McNulty, Midland, VA (US)

(21) Appl. No.: 13/546,085

(22) Filed: Jul. 11, 2012

Related U.S. Application Data
(60) Provisional application No. 61/506,840, filed on Jul. 12, 2011.

(51) Int. Cl. A61F 13/62; A61F 13/56; A61F 13/45

(52) U.S. Cl. 604/385.03; 604/385.01

(57) ABSTRACT

An absorbent pouch for use by males suffering from mild incontinence problems includes an outer layer or sheath made from an impermeable material and an inner liner made from an absorbent material. The outer layer is defined by first and second side wall portions which are interconnected along three edges so as to establish a single opening into the pouch. The first or inner side wall portion of the pouch is also formed with an arcuate cut-out portion leading into the pouch and provided with a soft material. Extending from the second or outer side wall portion of the pouch is a support tab provided with one or more fastening elements for securing the pouch below a waist portion of a user, such as by attaching the support tab to a waist band or an undergarment of the user.

### CROSS-REFERENCE TO RELATED APPLICATIONS

The present application claims priority to U.S. Provisional Patent Application Ser. No. 61/506,840 filed on Jul. 12, 2011 entitled “Disposable Incontinence Pouch Assembly.”

### BACKGROUND OF THE INVENTION

1. Field of the Invention

The present invention pertains to the field of medicine and, more particularly, to a lined, fluid absorbent, disposable pouch and mounting arrangement designed to address incontinence problems experienced by males.

2. Discussion of the Prior Art

Male incontinence can be generally defined as the uncontrolled leakage of urine from a penis. In extreme incontinence situations, a catheter will generally be employed as a link between a male urinary track and a urine collection bag. Such an arrangement is fairly common with bedridden or certain handicapped males. However, there also exist abundant situations of mild incontinence wherein the uncontrolled leakage of only small amounts of urine are experienced. For instance, a significant percentage of men who have prostate trouble, particularly following prostate surgery or other treatment, find themselves with the problem of uncontrollable leakage issues. Even when the problem only amounts to the dribbling of urine, the situation can be quite uncomfortable and, often times, embarrassing.

For quite some time, mainly to address the circumstances surrounding the relatively mild cases of incontinence, there were made available diapers or large pads designed to be worn under a user's trousers. Simple stated, these diapers or pads fool nobody as to their use by an individual, particularly based on baggy appearance and the sounds made when walking, sitting and the like by the wearer. For at least these reasons, these products merely deflect the potential embarrassment from one factor to another. It has also been proposed to secure a small pad with an absorbent liner, such as one similar to a common feminine pad, to a man's undergarment for the collection of the leakage. Unfortunately, the male organ has a tendency to shift significantly throughout the day, particularly with movement of the user. Therefore, these types of devices are simply not considered to be practical.

Perhaps most significant, the concept of providing a small pouch has been presented, including pouches which are secured directly about a user's penis, attached to existing undergarments or separately supported on the user. In each of these arrangements, a pouch is established into which a user's penis is positioned such that, should urinary leakage occur, the leakage will be confined to within the pouch. Given the reduced size required in connection with the pouch concept, these devices can be utilized to a much greater extent without being readily discoverable by others. In this sense, these types of devices can provide a user with a fair amount of self-assurance and a degree of comfort. However, in connection with the directly secured pouches, these pouches are thought to require considerable adjustment to assure their effectiveness based on size and position fluctuations of the male organ over a given period of use. Although attaching a pouch to an existing undergarment can provide the user a further level of comfort, this is a costly option, particularly considering that these mild incontinence issues may only be experienced for a relatively short period of time following prostate or other treatments.

With the above in mind, employing a relatively small pouch and attaching the pouch through a separate support arrangement is considered to be the most viable and desirable. Even though this broad concept has been previously proposed, there still exist significant shortcomings in known arrangements which have prevented them from being commercially accepted. In fact, even though there are various previously proposed arrangements of this type, the main available products to address the problem of incontinence still remain to be pads and diapers.

### SUMMARY OF THE INVENTION

The present invention is directed to an absorbent pouch for use by males suffering from mild incontinence problems, such as those often experienced after prostate surgery or other medical procedures. In accordance with the invention, the pouch includes an outer layer or sheath made from an impermeable material and an inner liner made from an absorbent material. More specifically, the outer layer is defined by first and second side wall portions which are interconnected along three edges so as to establish a single opening into the pouch. The first or inner side wall portion of the pouch is also formed with an arcuate cut-out portion leading into the pouch. The edge of the arcuate cut-out portion is provided with a soft material, preferably a concentrated portion of the absorbent material of the inner liner. Extending from the second or outer side wall portion of the pouch is a support tab provided with one or more fastening elements, such as a hook and loop-type pad.

For use in combination with the pouch, the overall assembly also includes an additional fastening element, such as a complimentary hook and loop-type pad. This additional fastening element can be attached to an inner surface portion of a user's boxer shorts or corresponding undergarment but, in accordance with the most preferred embodiment of the invention, the complimentary fastening element is provided on an adjustable, elastic waist band or belt member which is adapted to extend about a user's waist. With this arrangement, the band member can be secured about a user's waist, with the complimentary fastening element being centrally located relative to and exposed away from the user's body. Thereafter, the pouch can be attached to the band member by interconnecting the fastening elements such that the pouch extends directly in front of the user, with the arcuate cut-out portion being against the user. At this point, the user positions his penis in the pouch, with the head and a majority of the shaft portion of the penis extending therein, while a portion of the shaft portion of the user's penis will engage the soft material to provide comfort and protection during movement of the user while wearing the pouch. If desired, a pair of boxer shorts or other undergarment can be worn over the overall pouch assembly.

Once properly placed in use, if the user experiences any uncontrolled leakage, the urine will be absorbed by the inner liner and cannot seep through the fluid impermeable outer layer. Once used, the pouch can be easily detached from the waist band member. In accordance with a preferred embodiment of the invention, the pouch is provided with a further complimentary fastener element on a lower section of the first side wall portion, enabling the pouch to be folded upon itself a first time and then the tab can folded thereover such that the fastening element on the tab is engaged with the further complimentary fastener element to close off the pouch for disposal.

Additional objects, features and advantages of the present invention will become more readily apparent from the following detailed description of preferred embodiments when taken in conjunction with the drawings wherein like reference numerals refer to corresponding parts in the several views.

### BRIEF DESCRIPTION OF THE DRAWINGS

FIG. 1 is a perspective view showing an incontinence pouch constructed in accordance with a first embodiment of the present invention worn by a user;

FIG. 2 is a rear elevational view of the pouch of FIG. 1;

FIG. 3 is a front elevational view of the pouch of FIG. 1;

FIG. 4 depicts an elongated strip of manufactured material from which a series of the pouches of FIGS. 1-3 are separated during a formation process;

FIG. 5 illustrates an initial stage in folding the pouch of FIGS. 1-3 for disposal purposes;

FIG. 6 illustrates an subsequent stage in the folding of the pouch;

FIG. 7 is a rear elevational view of a pouch constructed in accordance with a second embodiment of the invention; and

FIG. 8 is a front elevational view of the pouch of FIG. 7.

### DETAILED DESCRIPTION OF THE PREFERRED EMBODIMENT

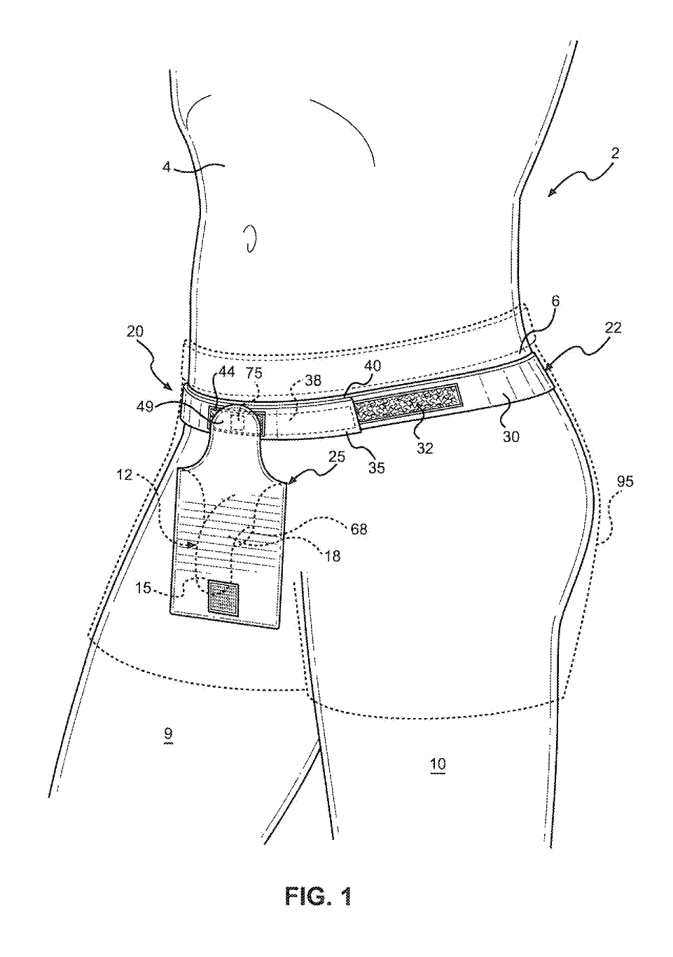

With initial reference to FIG. 1, a male is generally indicated at 2. As shown, male 2 includes a torso 4, a waist 6, legs 9 and 10, and a penis 12. As indicated, penis 12 includes a head portion 15 and a shaft portion 18. Male 2 is shown wearing a disposable incontinence pouch assembly 20 constructed in accordance with the present invention due to an existing, mild incontinence problem, such as experienced after prostate surgery or other medical procedure. Assembly 20 is shown to include a band or belt member 22 which is wrapped around waist 6 of male 2. Band member 22 can take various forms in accordance with the invention, including a leather, vinyl or cloth strap. However, in accordance with the most preferred form of the invention, band member 22 is constituted by an adjustable, elastic band to which is attached a pouch 25 of assembly 20.

In the embodiment shown, band member 22 includes an outer surface 30 upon which is mounted an elongated fastening element 32 which preferably takes the form of a hook and loop-type (VELCRO) fastener. Band member 22 also includes a terminal end section 35 which also has attached thereto a hook and loop-type fastening element 38 on an inner surface 40 thereof. With this arrangement, band member 22 can be adjustably secured about waist 6. In this preferred embodiment, band member can be stretched and attached with fastening element 38 being attached to fastening element 32 with a desired degree of comfort. Band member 22 also has provided, such as along terminal end section 35, an external fastening element 44 which may also take a form of a hook and loop-type fastening element. Pouch 25 is shown to include a support tab 49 which, as will be discussed more fully below, is removably attached to fastening element 44 and supports pouch 25 from band member 22.

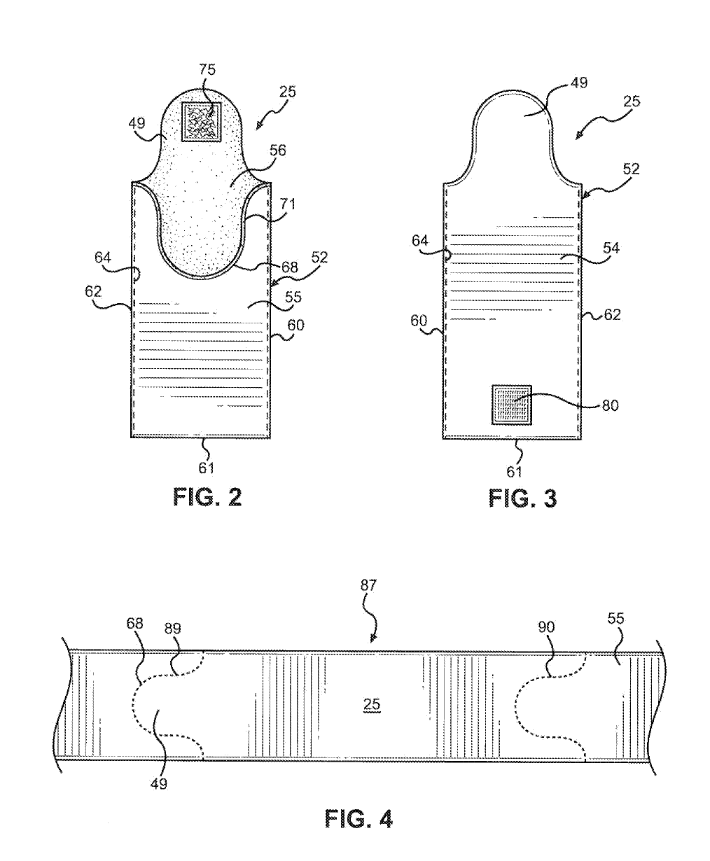

As perhaps best shown in FIGS. 2 and 3, pouch 25 includes an outer layer or sheath 52 having a first side wall portion 54 and a second side wall portion 55. Arranged between first and second side walls portions 54 and 55 is an inner liner 56. At this point, it should be noted that outer layer 52 is made from a fluid impermeable material and inner liner 56 is made from a soft, fluid absorbent material such as cotton. In the embodiment shown, pouch 25 has sealed peripheral edges 60-62 which are interconnected. Most preferably, pouch 25 is formed by folding second side wall portion 55 upon first side wall portion 54 to establish edge 61 and, thereafter, edges 60 and 62 are interconnected through a subsequent fastening process, such as through the use of either internal or external stitching 64 or heat sealing. As best shown in FIG. 2, second side wall portion 55 includes an arcuate, cut-out portion 68. Arcuate portion 68 has an edge 71 which is provided with additional soft material, such as additional material from inner liner 56, for reasons which will be discussed more fully below. As also shown in these figures, support tab 49 has attached thereto a fastening element 75, preferably again a hook and loop-type fastener, and a further fastening element 80 is attached on a lower section of first side wall portion 54.

At this point, it should be realized that pouch 25 can be formed in various ways. In accordance with the most preferred form of the invention, support tab 49 is advantageously complementary in shape to arcuate portion 68. More particularly, the first and second side wall portions 54 and 55 can be made by producing an elongated strip, shown at 87 in FIG. 4, from which can be made a series of pouches 25 by cutting strip 87 along perforations 89 and 90. With the cutting of each perforation 89, 90, the support tab 49 of one pouch 25 is formed at the same time as the arcuate portion 89 of another pouch 25. Although the particular materials employed can vary, in certain embodiments, sheath 52 and inner liner 56 are made of material directly corresponding to known hospital bed pads or baby diapers such as those including an inner liner material which turns to a gel upon becoming wet.

With reference back to FIG. 1, in use, pouch 25 is secured to band member 22 with fastening element 75 of support tab 49 engaging fastening element 44 in order to have pouch 25 hang directly in front of male 2, with arcuate, cut-out portion 68 being arranged against male 2. At this point, male 2 positions his penis 12 in pouch 25, with head portion 15 and a majority of shaft portion 18 of penis 12 extending therein. As clearly shown in this figure, shaft portion 18 will enter pouch 25 at arcuate portion 68. During movements by male 2, penis 12 will shift and be caused to abut arcuate portion 68. However, given the provision of additional soft material provided about edge 71 of arcuate portion 68, male 2 will be provided with comfort and protection during such movements. Although not required, a pair of boxer shorts 95 or other similar undergarment can be worn by male 2 over the overall assembly 20.

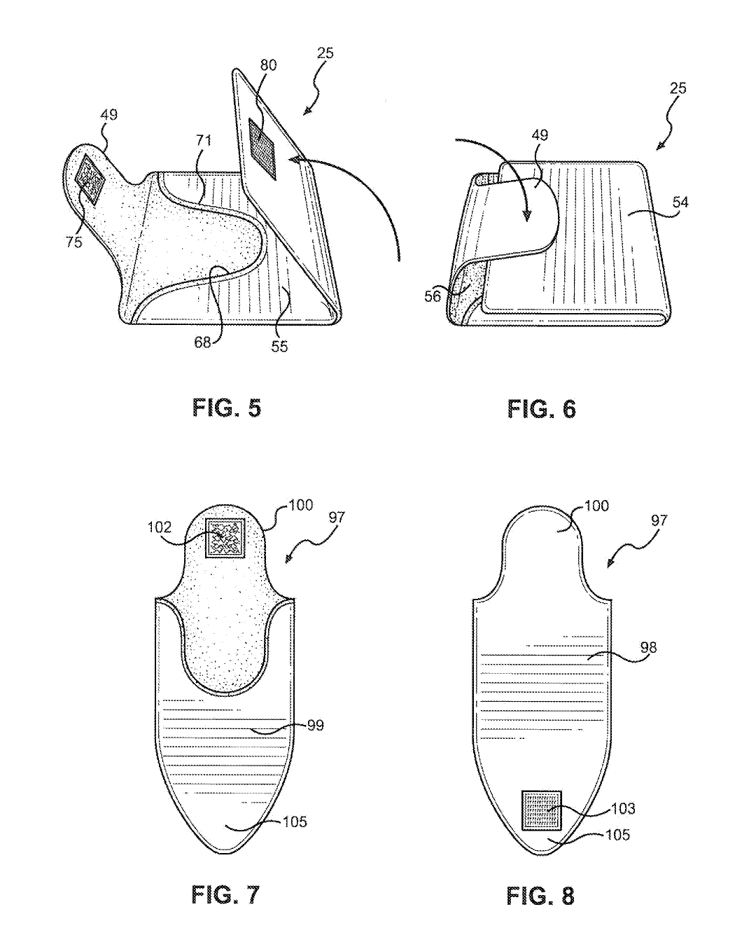

Once band member 22 and pouch 25 are properly supported with penis 12 therein, if male 2 experiences any uncontrolled leakage, the leaked urine will be readily absorbed by inner liner 56 and prevented from seeping through the impermeable outer layer 52. When it is convenient for male 2 to get to a restroom, pouch 25 can be readily removed by detaching fastening element 75 from fastening element 44. Thereafter, pouch 25 can be folded upon itself as best shown in FIG. 5, thereby arranging fastening element 80 adjacent fastening element 75. Next, support tab 49 can be folded (see FIG. 6) to interengage fastening element 75 to fastening element 80. In this condition, pouch 25 can be readily disposed of and a new pouch 25 can be placed on band member 22.

Although described with reference to preferred embodiments of the invention, it should be readily understood that various changes and/or modifications can be made to the invention without departing from the spirit thereof. For instance, the pouch of assembly 20 can take various different shapes and constructions without departing from the invention. For example, FIGS. 7 and 8 illustrate another embodiment of the invention wherein a pouch 97 is defined by side wall portions 98 and 99, a support tab 100, an arcuate portion 101, fastening elements 102 and 103 in a manner directly analogous to that described above. However, as shown, pouch 97 is provided with a more pointed or rounded lower section 105 which has been found to further limit relative shifting between penis 12 and pouch 97. In addition, it should be noted that the pouch of the invention could be used without the band member by providing a corresponding fastening element directly on a user's undergarment for attachment of the pouch, or even providing a specifically constructed undergarment which receives the pouch in a removably supporting manner. Furthermore, other types of fasteners, such as buttons, clips, clamps, snaps and the like, could also be employed. In any case, it should be readily apparent that the disposable incontinence pouch assembly of the invention provides a user with a highly comfortable, extremely convenient and nearly undetectable protection arrangement for mild incontinence problems. In any case, the invention is only intended to be limited by the scope of the following claims.

1. A disposable incontinence pouch comprising:
an outer sheath made from an impermeable material and defined by first and second side wall portions which are interconnected along three edges so as to establish a single opening into the pouch, said first side wall portion being formed with an arcuate cut-out portion having an edge provided with a soft material; and
an inner liner made from an absorbent material.

2. The pouch according to claim 1, wherein the soft material provided at the cut-out portion constitutes a concentrated portion of the absorbent material of the inner liner.

3. The pouch according to claim 2, further comprising: a support tab extending from the second side wall portion of the pouch.

4. The pouch according to claim 3, wherein the support tab is provided with one or more first fastening elements for securing the pouch to a user.

5. The pouch according to claim 4, wherein the one or more first fastening elements are provided on a hook and loop-type pad.

6. The pouch according to claim 5, further comprising: one or more second fastening elements provided at a position spaced from the one or more first fastening elements, wherein the one or more first and second fastening elements are arranged and configured to be interconnected upon folding the pouch.

7. The pouch according to claim 6, wherein the one or more first and second fastening elements are provided on opposing sides of the pouch.

8. The pouch according to claim 6, further comprising: a band member adapted to be worn about a waist of a user of the pouch, wherein the one or more second fastening elements are provided on the band member.

9. The pouch according to claim 1, wherein the pouch is provided along a strip including a plurality of interconnected said pouches.

10. A disposable incontinence pouch comprising:
an outer sheath made from an impermeable material and defined by first and second side wall portions which are interconnected along three edges so as to establish a single opening into the pouch;
an inner liner made from an absorbent material; and
a support tab extending from the second side wall portion of the outer sheath for attaching the pouch to a waist region of a user.

11. The pouch according to claim 10, further comprising: a band member adapted to be worn about a waist region of a user of the pouch, said support tab being configured to be attached to the band.

12. The pouch according to claim 11, further comprising: an arcuate cut-out portion formed on the first side wall portion, said arcuate cut-out portion including an edge provided with a soft material.

13. The pouch according to claim 12, wherein the soft material provided at the cut-out portion constitutes a concentrated portion of the absorbent material of the inner liner.

14. The pouch according to claim 11, wherein the support tab is provided with one or more first fastening elements for securing the pouch to a user.

15. The pouch according to claim 14, wherein the one or more first fastening elements are provided on a hook and loop-type pad.

16. The pouch according to claim 15, further comprising: one or more second fastening elements provided at a position spaced from the one or more first fastening elements, wherein the one or more first and second fastening elements are arranged and configured to be interconnected upon folding the pouch.

17. The pouch according to claim 16, wherein the one or more first and second fastening elements are provided on opposing sides of the pouch.

18. The pouch according to claim 10, wherein the pouch is provided along a strip including a plurality of interconnected said pouches.

19. A method of addressing incontinence of a male comprising:
hanging a disposable incontinence pouch, including an outer sheath made from an impermeable material and defined by first and second side wall portions which are interconnected along three edges so as to establish a single opening into the pouch, said first side wall portion being formed with an arcuate cut-out portion having an edge provided with a soft material, and an inner liner made from an absorbent material, below a waist region of a user; and
inserting a penis of a user into the pouch at the cut-out portion, with the penis engaging the soft material, wherein the absorbent material can absorb leaking urine.

20. The method of claim 19, further comprising: folding the pouch and securing the pouch in a folded condition through at least one fastener provided on the pouch for disposal.
